(19) United States
(12) Patent Application Publication
(10) Pub. No.: US 2010/0304471 A1
(43) Pub. Date: Dec. 2, 2010

(54) ASSAY

(76) Inventors: Jun Tao, Poway, CA (US); Helen Grace Huang, San Diego, CA (US)

Correspondence Address: DLA PIPER LLP (US), 4365 EXECUTIVE DRIVE, SUITE 1100, SAN DIEGO, CA, 92121-2133, US

(21) Appl. No.: 12/739,388

(22) PCT Filed: Dec. 1, 2008
(86) PCT No.: PCT/US08/85209

§ 371 Date: Aug. 16, 2010

Related U.S. Application Data
(60) Provisional application No. 60/991,098, filed on Nov. 29, 2007.

(51) Int. Cl. C12M 1/34; G01N 33/48

(52) U.S. Cl. 435/287.2; 422/68.1

(57) ABSTRACT

The present invention relates to test devices, and in particular devices capable of detecting the presence or absence of an analyte in a sample, such as a liquid sample. Also provided are methods of using such devices for quantitative or qualitative measurement of one or more analytes in a liquid sample.

### CROSS-REFERENCE

This application claims the benefit of U.S. Provisional Application No. 60/991,098, filed Nov. 29, 2007, which application is incorporated herein by reference in its entirety.

### BACKGROUND

Assays can be performed for determining one or more analytes in a sample simultaneously or separately. Assays can be quantitative in that they provide a readout for the amount or concentration of the analyte present in a sample. Alternatively, or in combination, assays can be qualitative in that the result is indicative of the presence or absence of a given analyte.

A typical lateral flow assay device includes a porous sample receiving pad, a conjugate pad in liquid communication with the receiving pad, and a test strip in liquid communication with the conjugate pad. Sometimes, the conjugate pad contains dry conjugates including particles labeled with an antibody for the analyte. The test strip has a detection zone that contains, in an immobilized form, the analyte or an analyte conjugate capable of binding the analyte. In practice, a liquid sample is applied to a receiving pad. The sample travels to a conjugate pad where it mobilizes the dry conjugates. The sample with mobilized conjugates travels to the test strip and reaches the detection zone, allowing for the presence or absence of analyte in the liquid sample to be determined. See, e.g. U.S. Pat. Nos. 5,451,504, 5,707,818, 6,121,008, 6,699,722, and 7,393,697.

### SUMMARY OF THE INVENTION

Conventional devices and methods for performing lateral flow assays samples are not particularly amenable to adjusting detection thresholds. In particular, these devices and methods are not particularly useful for measuring analytes at high concentrations with high degree of accuracy as a result of, e.g., a hook effect.

In one embodiment, the present invention provides a test device for detecting an analyte in a liquid sample. The test device typically comprises a matrix for supporting the liquid sample flowing thereon, wherein the matrix comprises: (a) a blocking zone comprising a blocking-zone binding agent immobilized thereon, wherein said analyte in said liquid sample and a conjugate comprising an analyte mimic when present compete for binding to said blocking-zone binding agent, wherein binding affinity between the analyte mimic and the blocking-zone binding agent is higher than that between the analyte and the blocking-zone binding agent; and (b) a detection zone comprising immobilized thereon a detection-zone binding agent that exhibits binding specificity to a conjugate comprising said analyte mimic.

In one aspect, the conjugate further comprises a detectable label and the analyte mimic, said label and mimic are linked via a linker. Where desired, the linker can be selected from the group consisting of keyhole limpet hemocyanin (KLH), bovine gamma globulin (BGG), bovine serum albumin (BSA), bovine thyroglobulin (BTG), hen egg-white lysozyme (HEL), ovalbumin (OVA), sperm whale myoglobin (SWM), tetanus toxoid (TT), methylated bovine serum albumin (mBSA), and rabbit serum albumin (RSA).

In another aspect, the binding affinity between the analyte mimic and the blocking-zone binding agent is at least 1, 2, 3, 4, 5, 10, 50, 100 fold higher than the binding affinity between the analyte and the blocking-zone binding agent, as measured by association constants.

In yet another aspect, the blocking zone comprises a plurality of distinct blocking-zone binding agents, individual members of said plurality exhibit binding specificity to distinct analytes present in said sample.

In still another aspect, the detection zone comprises a plurality of distinct detection-zone binding agents, individual members of said plurality exhibit binding specificity to distinct conjugates present in said sample. Where desired, each distinct conjugate comprises a detectable label and said analyte mimic, wherein said detectable label and said analyte mimic are linked via a linker, and further wherein the distinct conjugates are differentiated by one or more members selected from the group consisting of distinct linkers, distinct analyte mimics, and distinct detectable labels.

In some aspects, the distinct blocking-zone binding agents are each immobilized to distinct regions.

In some aspects, the distinct detection-zone binding agents are each immobilized to distinct regions.

Where desired, the detectable label comprises a colored particle. The blocking-zone binding agent and/or the detection-zone binding agent can be an antibody.

One illustrative design of the subject device comprises a matrix having a mobilization zone, said mobilization zone comprising the conjugate having a detectable label and the analyte mimic, wherein the conjugate is mobilizable upon application of said liquid sample. The mobilization zone comprises a plurality of distinct conjugates, wherein members of said plurality exhibit binding specificity to distinct blocking-zone binding agents and/or distinct detection-zone binding agents. The matrix can be a porous membrane.

In a separate but related embodiment, the present invention provides a test device for detecting an analyte in a liquid sample. The device comprises a matrix for supporting the liquid sample flowing thereon, wherein the matrix comprises (a) a blocking zone comprising a blocking-zone binding agent, wherein the blocking-zone binding agent comprises an analyte mimic immobilized on the blocking zone, wherein said analyte in said liquid sample and said analyte mimic compete for binding to a conjugate when present, wherein binding affinity between the analyte mimic and the conjugate is higher than that between the analyte and the conjugate; and (b) a detection zone comprising, immobilized thereon, a detection-zone binding agent that exhibits binding specificity to said conjugate.

In one aspect, the conjugate of this design further comprises a detectable label and a moiety that exhibits binding specificity to said analyte and said analyte mimic, wherein said label and said moiety are linked via a linker.

In another aspect, the blocking zone comprises a plurality of distinct analyte mimics, individual members of said plurality of analyte mimics exhibit binding specificity to distinct conjugates present in said sample. Where desired, the detection zone comprises a plurality of distinct detection-zone binding agents, individual members of said plurality exhibit binding specificity to distinct conjugates present in said sample.

In yet another aspect, each distinct conjugate comprises a detectable label and the moiety, said detectable label and said moiety are linked via a linker, and further wherein the distinct conjugates are differentiated by one or more members selected from the group consisting of distinct linkers, distinct moieties, and distinct detectable labels.

In still another aspect, the distinct analyte mimics are each immobilized to distinct regions.

In other aspects, the distinct detection-zone binding agents are each immobilized to distinct regions. Where desired, the detectable label comprises a colored particle.

The linkers for use in this design can be selected from the group consisting of keyhole limpet hemocyanin (KLH), bovine gamma globulin (BGG), bovine serum albumin (BSA), bovine thyroglobulin (BTG), hen egg-white lysozyme (HEL), ovalbumin (OVA), sperm whale myoglobin (SWM), tetanus toxoid (TT), methylated bovine serum albumin (mBSA), and Rabbit Serum Albumin (RSA).

Where desired, the moiety and/or the detection-zone binding agent are an antibody.

In one illustrative design, the matrix comprises a mobilization zone, said mobilization zone comprising the conjugate having a detectable label and the moiety, wherein the conjugate is mobilizable upon application of said liquid sample. The binding affinity between the analyte mimic and the conjugate is at least 1, 2, 3, 4, 5, 10, 50, 100 fold higher than that between the analyte and the conjugate, as measured by association constants.

The present invention also provides a method for detecting an analyte in a liquid sample. The method typically comprises (a) applying said liquid sample to a test device of the present invention to effect competitive binding of said analyte and said conjugate for said blocking-zone binding agent; and (b) determining the presence of said conjugate bound to said blocking-zone binding agent and/or said detection-zone binding agent, thereby detecting the presence of said analyte in said liquid sample.

In one aspect, the step of determining comprises measuring the amount of conjugate bound to said blocking-zone binding agent and/or said detection-zone binding agent, thereby quantifying the analyte in said liquid sample. In another aspect, the step of determining comprises detecting the amount of conjugate bound to said detection-zone binding agent. In yet another aspect, the step of determining comprises detecting the amount of conjugate bound to said blocking-zone binding agent. The method may also comprises the step of determining the presence of a plurality of distinct conjugates bound to said blocking-zone, thereby detecting the presence of a plurality of distinct analytes in said liquid sample. Where desired, the distinct conjugates are detected in distinct regions. The matrix can comprise a mobilization zone having said conjugate, wherein said conjugate is mobilized upon applying said liquid sample to the test device. Alternatively, the conjugate can be added to the liquid sample prior to applying the liquid sample to the test device.

In a separate but related embodiment, the present invention provides a method for detecting an analyte in a liquid sample, which comprises the steps of (a) applying said liquid sample to a test device of the present invention to effect competitive binding of said analyte and said conjugate for said blocking-zone binding agent; (b) determining the presence of said conjugate bound to said blocking-zone binding agent and/or said detection-zone binding agent, thereby detecting the presence of said analyte in said liquid sample.

In one aspect, the step of determining may comprise measuring the amount of conjugate bound to said blocking-zone binding agent and/or said detection-zone binding agent, thereby quantifying the analyte in said liquid sample. In another aspect of this method, the step of determining comprises detecting the amount of conjugate bound to said detection-zone binding agent. In yet another aspect of this method, the step of determining comprises detecting the amount of conjugate bound to said blocking-zone binding agent. Where desired, this method may further comprise the step of determining the presence of a plurality of distinct conjugates bound to said blocking-zone, thereby detecting the presence of a plurality of distinct analytes in said liquid sample. Any other variations mentioned for the related method above can be applied as well.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 is a perspective view of a lateral flow device.

FIG. 2A is a perspective view of a lateral flow device showing a conjugate.

FIG. 2B is a perspective view of a lateral flow device showing a conjugates in the in the absence of an analyte.

FIG. 2C is a perspective view of a lateral flow device showing a conjugates in the in the presence of an analyte.

FIG. 3 is an illustration of a conjugate with an analyte mimic, a linker agent, and a label.

FIG. 4 is an illustration of a lateral flow device where the affinity between a receptor in the first capture zone and a labeled ligand is lower than the affinity between the receptor and an analyte.

FIG. 5 is an illustration of a lateral flow device where the affinity between a receptor in the first capture zone and a labeled ligand is higher than the affinity between the receptor and an analyte.

FIG. 6 is an illustration of a lateral flow device configured for assaying MOR.

FIG. 7 is an illustration of a lateral flow device configured for assaying COC.

FIG. 8 is an illustration of a lateral flow device configured for assaying THC, COC, and MOR.

FIG. 9 is an illustration of a lateral flow device configured for assaying MOR or COC.

FIG. 10 is an illustration of a lateral flow device configured for assaying THC, COC, and MOR, where the first capture zone includes antibodies.

FIG. 11 is an illustration of a lateral flow device configured for assaying COC, where the first capture zone includes analyte analogues.

FIG. 12 is an illustration of a lateral flow device configured for assaying COC and MOR, where the first capture zone includes analyte analogues.

FIG. 13 is an illustration showing THC and THC metabolites.

### DETAILED DESCRIPTION

### Devices of the Present Invention

Assays for detecting one or more analytes in a sample are described. While preferable embodiments of the invention have been shown and described herein, it will be obvious to those skilled in the art that such embodiments are provided by way of example only. The devices and/or methods of the invention can be employed individually or combined with other devices, methods, and/or systems in manners know to those skilled in the arts for detection of one or more analytes in a sample.

The present invention relates to assays, such as assays for determining drugs of abuse in biological samples. As used herein, the term “assay” or “assays” encompasses both assay device(s) or an assay method(s) unless stated to the contrary.

As used in the specification and claims, the singular form “a”, “an” and “the” include plural references unless the context clearly dictates otherwise.

In some embodiments, the assays are lateral flow assays. Typically, the lateral flow devices have a flow path which includes a first blocking capture zone and a detection capture zone for each analyte to be detected. During operation, a liquid sample is applied to the flow path of the device. The liquid first flows along the flow path to the blocking capture zone(s) (or blocking zone(s)) and then to the detection capture zone(s) (or detection zone(s)). If analyte is absent from the liquid sample, fewer or no detectable labels are captured in the corresponding detection capture zone. If analyte is present in the liquid sample, detectable labels are captured at the detection capture zone corresponding to that analyte. Thus, the presence of the detectable labels in the detection capture zone is indicative of the presence of the analyte in the sample.

In one embodiment of the invention, a test device for detecting an analyte in a sample comprises a matrix having a blocking capture zone and a detection capture zone. The blocking capture zone, or blocking zone herein, cap have a blocking-zone binding agent that is immobilized in or on the blocking capture zone. The blocking-zone binding agent can have an affinity to the analyte or an analyte mimic. The analyte and the analyte mimic can exhibit competitive binding or compete for binding to the blocking-zone binding agent. The affinity between the analyte mimic and the blocking-zone binding agent can be stronger or higher than the affinity between the analyte and the blocking-zone binding agent.

The matrix can be a porous surface or a non-porous surface. The porous surface can be a membrane, e.g. an absorbent material through which the test reagents flow. Paper or pulp products, glass fibers, or polymers, e.g. nitrocellulose, or nylon, can be used as absorbent materials of the devices described herein. In some embodiments of the invention, non-adsorbent materials are used as the matrix. The non-adsorbent materials can be configured to create a capillarity between two surfaces that allows for fluid movement along the assay device.

The matrix can provide for fluid communication between a plurality of zones in the device. These zones can be mobilization zones, blocking capture zones, and/or detection capture zones. The zones can be positioned in the same plane along the test device, or they can be placed above or below and/or to the side of each other. For example, the mobilization zone can be placed above the blocking capture zone and the blocking capture zone can be placed to the side of the detection capture zone. Any three-dimensional configuration of the zones can be utilized in the test devices described herein.

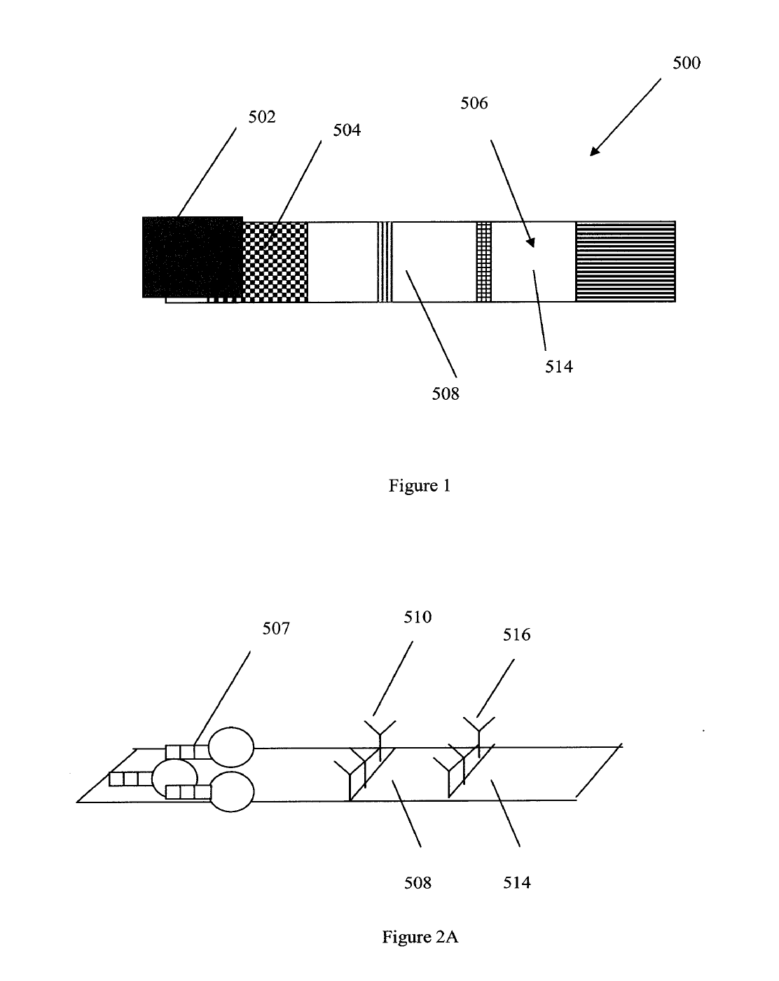

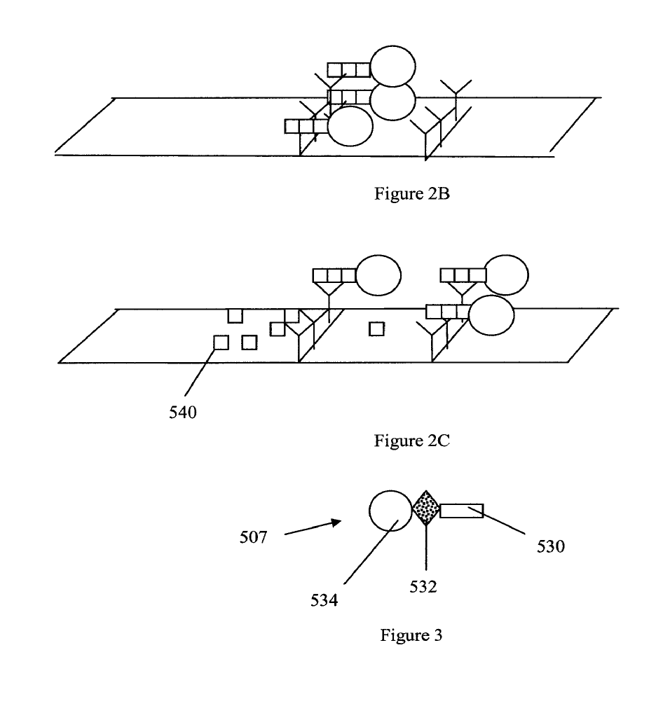

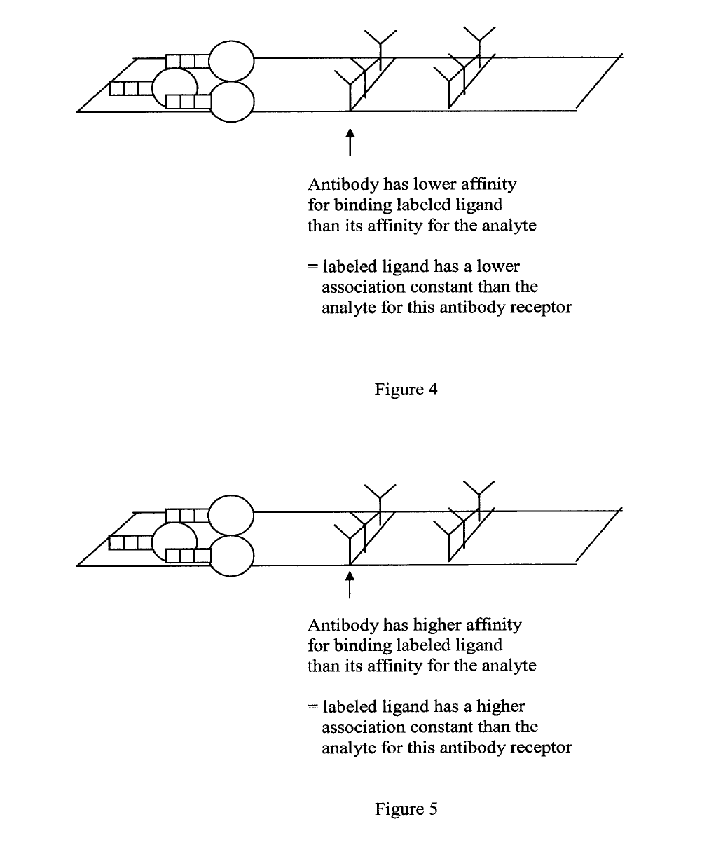

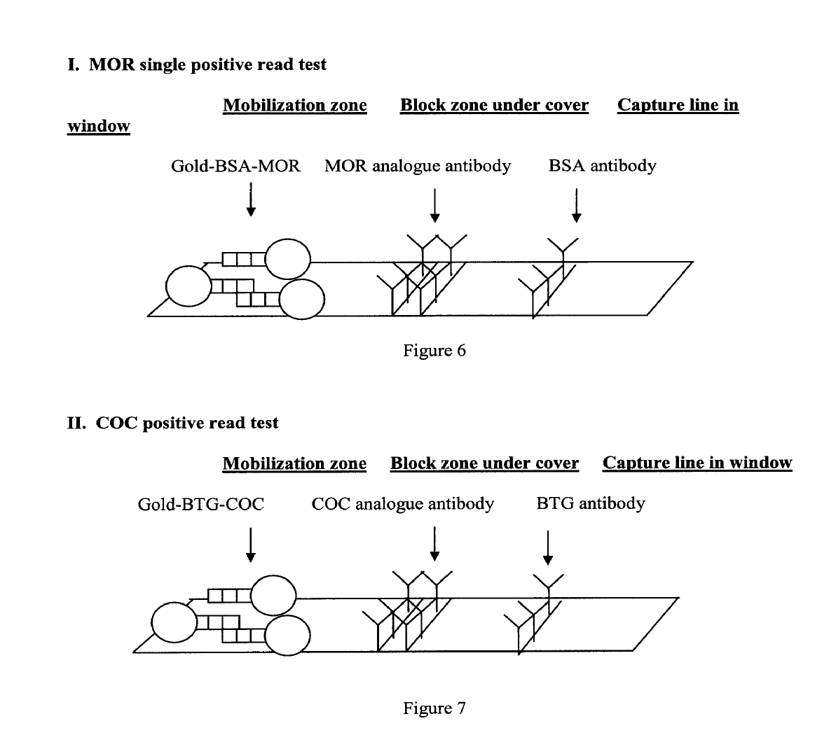

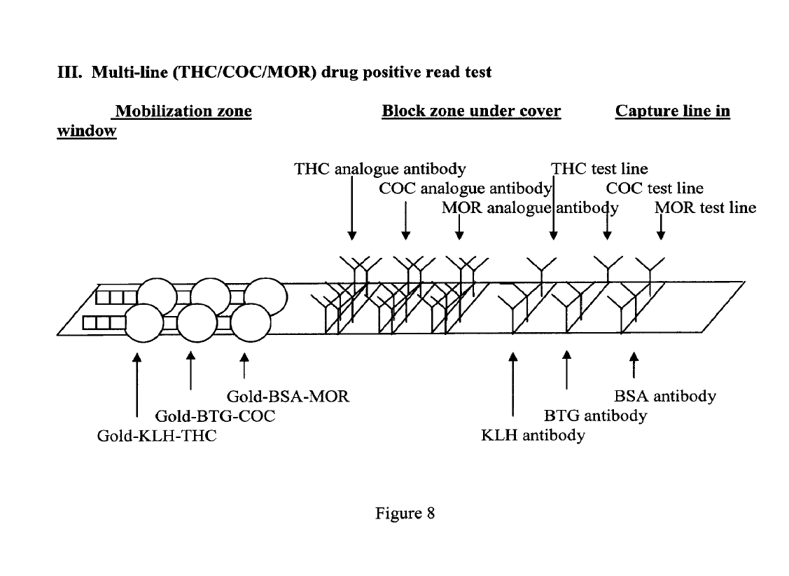

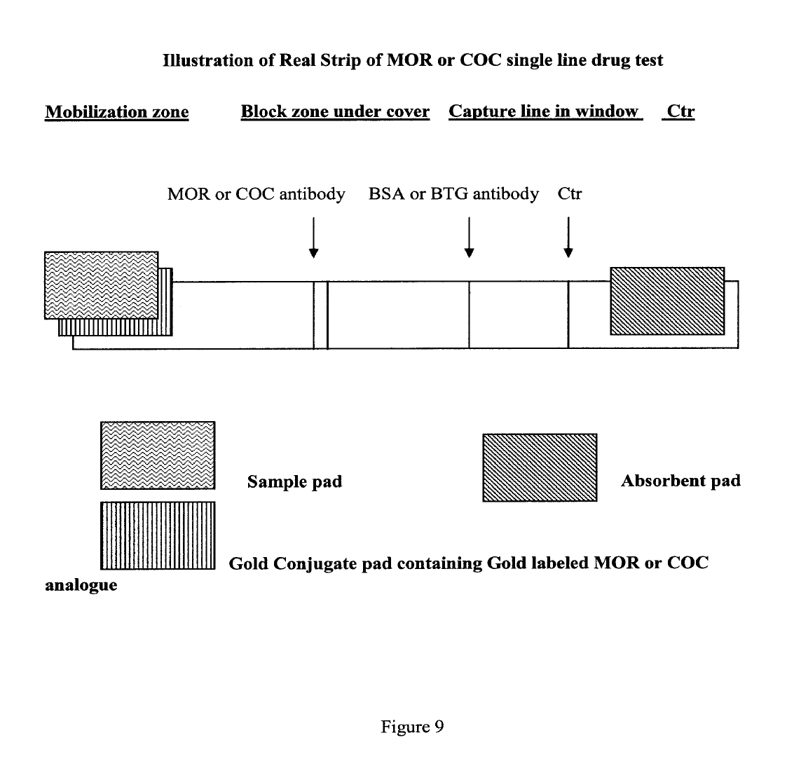

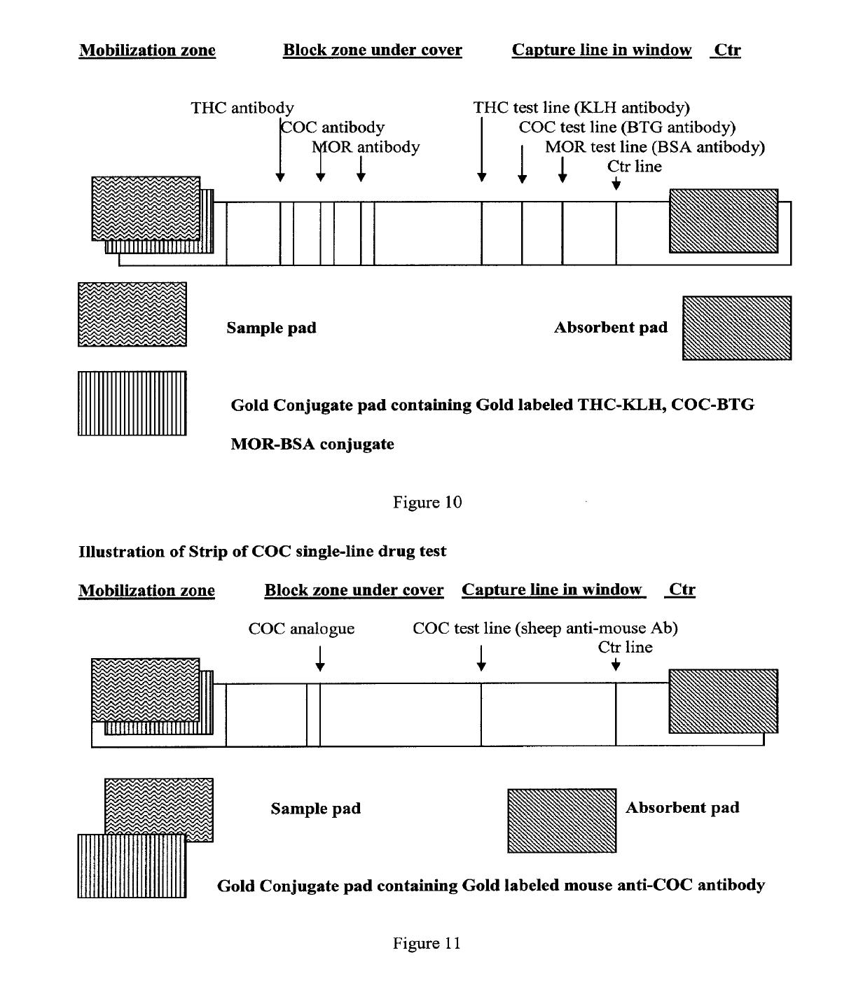

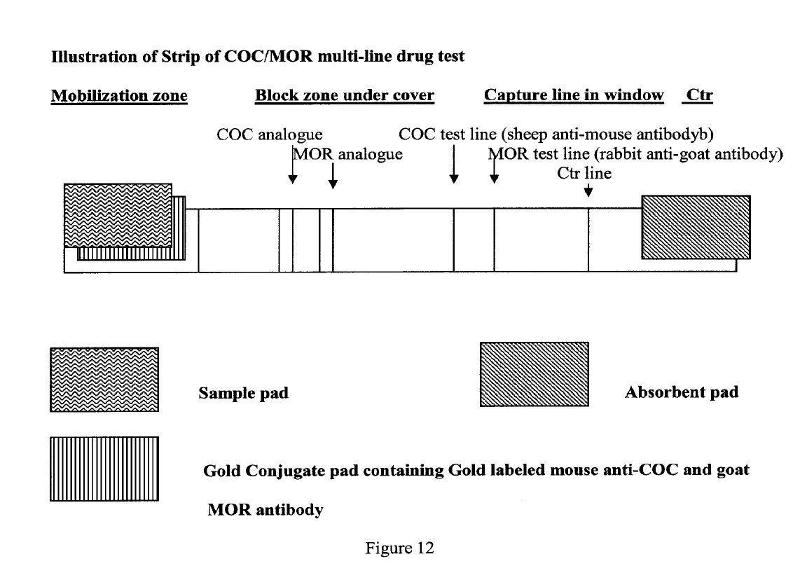

FIG. 1 and FIGS. 2a-2c, illustrate an exemplary lateral flow assay device 500 for detecting an analyte includes a sample receiving pad 502, a conjugate pad 504, and a flow strip 506. Conjugate pad 504 includes conjugates 507, which are typically in a dry state prior to use of assay device 500. Flow strip 506 includes a first capture zone 508 having binding agents 510 and a detection capture zone 514 having binding agents 516. Binding agents 510 are capable of binding conjugates 507 and are capable of binding the analyte. Thus, conjugates 507 and the analyte corresponding to conjugates 507 compete for binding to binding agents 510. Binding agents 516 are capable of binding conjugates 507. Binding agents 516 may have little or no affinity for analyte.

Flow strip 506 permits liquid to flow therealong and permits binding agents 510 and 516 to be immobilized with respect to strip 506. Typically, flow strip 506 is formed of a porous material such as nitrocellulose.

Referring back to FIG. 1, receiving pad 502 is capable of receiving a liquid sample. Typically, receiving pad 502 is formed of a porous material such as glass fiber.

Conjugate pad 504 retains conjugates 507 in a dry state and permits the liquid sample to mobilize conjugates 507. The mobilized conjugates flow with the sample along the flow path of device 500 to flow strip 506. Typically, conjugate pad 504 is formed of a porous material such as glass fiber.

Binding agents 510 are typically capable of specifically binding conjugates 507 (via analyte mimic 530) and are capable of binding the analyte corresponding to conjugates 507. By specifically binding it is meant that concomitants expected to accompany the analyte in the sample liquid do not interfere with the competition between conjugates 507 and the corresponding analyte for binding to binding agents 510. In exemplary embodiments, binding agents 510 are antibodies that recognize analyte mimic 530 and the corresponding analyte. For example, analyte mimic 530 and the corresponding analyte may each have an epitope that is recognized by the same antibody. Thus, the antibody will bind both the analyte mimic and the corresponding analyte.

Binding agents 510 are capable of binding both the analyte and conjugate 507. Binding agents typically bind conjugate 507 at least in part via analyte mimic 530. In exemplary embodiments, the binding affinity of binding agents 510 for conjugate 507 is higher than the binding affinity of binding agents 510 for the analyte that corresponds to analyte mimic 530. Typically, the affinity of binding agents 510 for conjugates 507 is higher than the affinity of binding agents 510 for the corresponding analyte.

Antibodies that can be used as binding agents can be any antibody known to those skilled in the art. Antibodies can be immunoglobulin molecules and antigen-binding portions of immunoglobulin molecules, i.e., molecules that contain an antigen binding site which specifically binds (“immunoreacts with”) an analyte, an analyte mimic, or a ligand. Antibodies also encompasses hybrid antibodies, or altered antibodies, and fragments thereof, including but not limited to, Fab fragment(s) and Fv fragment(s). The antigen-binding function of an antibody can be performed by fragments of a naturally-occurring antibody. Examples of binding fragments or antibody fragments include, but are not limited to, (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546 (1989)) which consists of a VH domain; (v) an isolated complimentarity determining region (CDR); and (vi) an F(ab′)₂ fragment, a bivalent fragment comprising two Fab fragments linked by a disulfide bridge at the hinge region. Furthermore, although the two domains of the Fv fragment are generally coded for by separate genes, a synthetic linker can be made that enables them to be made as a single protein chain (known as single chain Fv (scFv); Bird et al., Science 242:423-426 (1988); and Huston et al., PNAS 85:5879-5883 (1988)) by recombinant methods. Antibody fragments can include those which are capable of crosslinking their target antigen, e.g., bivalent fragments such as F(ab′)2 fragments. Alternatively, an antibody fragment which does not itself crosslink its target antigen (e.g., a Fab fragment) can be used in conjunction with a secondary antibody which serves to crosslink the antibody fragment, thereby crosslinking the target antigen.

Referring to FIG. 3, conjugates 507 include an analyte mimic 530, a linker agent 532, and a label 534. Binding between binding agents 510 and conjugates 507 takes place via analyte mimic 530 of conjugates 507.

Conjugates or binding conjugates 507 capable of binding the analyte are captured by binding agents 510 of first capture zone 508 through binding between the analyte mimic 530 and binding agents 510. However, an affinity of binding between binding agents 510 and conjugates 507 is higher than an affinity of binding between binding agents 510 and the analyte corresponding to conjugates 507. Analyte mimic 530 is capable of forming a complex with binding agents (e.g., an antibody) that also forms a complex with an analyte corresponding to the conjugate.

In some embodiments, the analyte mimic that corresponds to a particular analyte may be the analyte itself (e.g., analyte mimic 530 may be an analyte related to a drug as discussed above). For example, in an assay for marijuana, the analyte and analyte mimic may both be THC; for cocaine both may be benzoylecgonine; for morphine both may be morphine sulfate; and for amphetamine both may be amphetamine.

As an alternative to a drug or drug metabolite, analyte mimic 530 may be an analyte analogue that is capable of forming a complex with an antibody that also forms a complex with the analyte. For example, analyte mimic 530 may be a fragment of an analyte, the fragment retaining an epitope of the analyte.

Linker 532 links analyte mimic 530 and label 534. Typically, linker 532 has a binding site that is not present on the analyte, analyte mimic 530 or label 534. For example, the binding site may be an epitope capable of being recognized by an antibody that does not recognize either the analyte, analyte mimic 530 or label 534. In some embodiments, the binding site of such linkers is capable of being recognized by antibodies that also do not recognize the binding sites of other linkers that may be present. Examples, of such linkers include bovine serum albumin (BSA), keyhole limpet hemocyaninconjugate (KLH), and bivone benzoylecgonine (BBG), bovine thyroglobulin (BTG), hen egg-white lysozyme (HEL), ovalbumin (OVA), sperm whale myoglobin (SWM), tetanus toxoid (TT), methylated bovine serum albumin (mBSA), Rabbit Serum Albumin (RSA).

Label 534 permits detection of conjugate 507. In embodiments, label 534 is a particle (e.g., a latex particle, a metallic particle, or a colloidal particle). Such particles typically form a color when aggregated as at a detection capture zone. In some embodiments, label 534 is an enzyme label. Such enzymes can interact with a substrate to produce a detectable product such as a colored product. Other labels may also be used (e.g., radioactive labels).

A sample can be any material that is to be analyzed for the presence of an analyte of interest. The sample can be in liquid form. In exemplary embodiments, the liquid sample is a bodily fluid (e.g., urine, saliva, or a blood derived fluid such as whole blood, plasma, or serum). As described in more detail below, the sample may be a solid. However, in this case the sample can be solubilized or extracted prior to use in the test. The liquid sample may include additional materials such as a buffer solution.

An analyte can be any molecule or compound whose presence is to be identified in a sample. Analytes may include, without limitation, viral antigens, bacterial antigens, hormones, such as insulin, follicle stimulating hormone (FSH), thyrotropin, relaxin, somatotropin and gonadotropin, enzymes, immunoglobulins, cytokines, drugs, cancer antigens, antigenic polysaccharides, and nucleic acids. Alternatively, an analyte can be anti-HIV antibodies, anti-HCV antibodies and human chorionic gonadotropin (hCG).

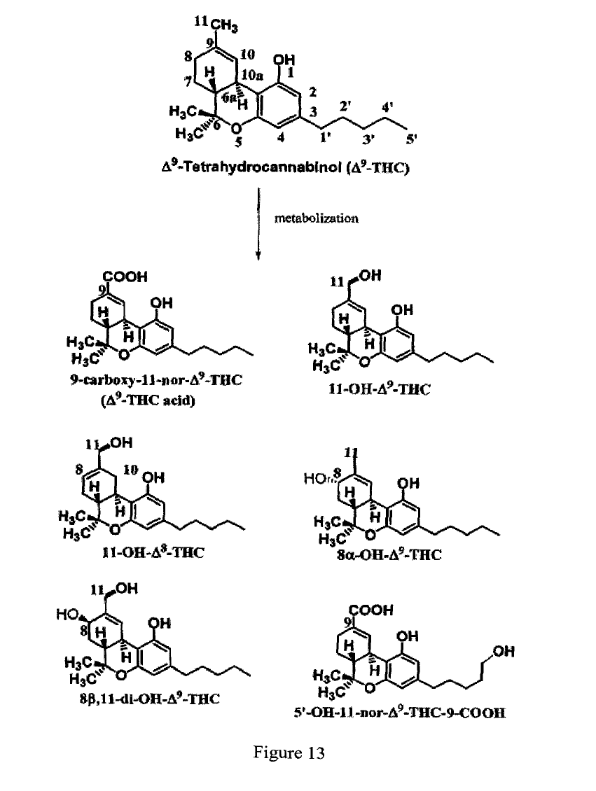

In exemplary embodiments, one or more of the analytes (if more than one is to be detected) is related to a drug (e.g., a drug of abuse). Detection of the analyte in a sample obtained from an individual correlates with consumption of the related drug by the individual. For some drugs, the analyte is a component of the drug itself and, for other drugs, the analyte is a metabolite of the drug. Examples of analytes related to a drug include 1 l-nor-Δ9-THC-9-COOH (THC) (related to marijuana), benzoylecgonine (cocaine), morphine sulfate (morphine), and amphetamine (amphetamine). An analyte can also be a metabolite of any molecule described herein. For example, an analyte can be a metabolite of THC, as shown in FIG. 13. Other analytes that may be identified in samples using the provided methods and apparatus will be apparent to one of ordinary skill in the art. As used herein, THC means tetrahydrocannabinol and analogues thereof, COC means cocaine and analogues thereof, and MOR means morphine and analogues thereof.

### Uses of the Devices of the Present Invention

As noted above, the present invention provides a method for detecting an analyte in a liquid sample using one or more of the subject devices. The method can be quantitative or qualitative. In one embodiment, the method comprises (a) applying said liquid sample to a test device of the present invention to effect competitive binding of said analyte and said conjugate for said blocking-zone binding agent; and (b) determining the presence of said conjugate bound to said blocking-zone binding agent and/or said detection-zone binding agent, thereby detecting the presence of said analyte in said liquid sample.

As an illustrative example, a lateral flow assay provides for determining the presence of an analyte in a sample and for showing positive read out. Sample is applied to move through three zones (mobilization zone, blocking capture zone and the detection capture zone).

1). Mobilization zone: a conjugate, for example a color labeled ligand formed by a protein (or enzyme or peptide or hapten or big MW molecular) conjugated with analogue (or analyte mimic) is conjugated with color-full particle in the mobilization zone.

2). Anti-analogue antibody coated on membrane in the blocking capture zone with higher affinity for binding color labeled ligand than its affinity for analyte.

3). Anti-protein (or enzyme or peptide or hapten or big MW molecular) antibody coated on membrane in the detection capture line.

When applying negative sample (analyte free), the color labeled ligand is captured by anti-analogue antibody in the blocking zone and unable to reach to the detection capture zone, so no color line showed in detection capture zone.

When applying positive sample (with analyte), the anti-analogue antibody in blocking zone is blocked by the analyte in the sample, and the color labeled ligand will be able to go through the blocking zone and will be captured by anti-protein (or enzyme or peptide or big MW molecular) antibody to form a color-full line in detection capture zone.

1). Color labeled ligands which are mobilized by and move with the sample have a higher association constant than the analyte for a receptor which is anti-analogue antibody coated on membrane as block zone.

2). The cut off level of drug test can be adjusted up to a desired level which is commonly used on market by selecting anti-analogue antibody with higher affinity for binding labeled ligand than its affinity for analyte.

3). This test can detect analyte at, no limit, any high concentration (no hook effect).

With reference to the illustrative figures, sample receiving pad 502, conjugate pad 504, and flow strip 506 are positioned and configured such that when a liquid sample is applied to sample receiving pad 502, the liquid travels along the flow path to conjugate pad 504. The advancing liquid mobilizes conjugates 507 and travels, with any analyte present and the mobilized conjugates, along the flow path to first capture zone 508 of flow strip 506. When the advancing liquid encounters binding agents 510 of first capture zone 508, analyte 540 (if present) and the conjugates 507 compete to form complexes with binding agents 510. Analyte or conjugates are captured by binding agents 510. As the amount of analyte in the liquid increases, the amount of conjugates captured by binding agents 510 decreases.

The advancing liquid continues along strip 506 past first capture zone 508 to detection capture zone 514. Conjugates 507 remaining in the liquid are captured by binding agents 516 as the liquid passes through detection capture zone 514. Because increasing amounts of analyte in the liquid sample result in an increasing amount of conjugates 507 passing through first capture zone 508, increasing amounts of analyte result in an increased amount of conjugates 507 being captured in detection capture zone 514. Thus, the assay device 500 is a positive-read assay device in that the presence (or presence above a threshold) of conjugates 507 in the detection zone indicates the presence of the analyte and the absence (or presence below a threshold) of the conjugates 507 indicates the absence of the analyte.

The presence of the detectable labels maybe indicated by, for example, the formation of a particular color in the detection capture zone. On the other hand, if analyte is present at lower levels or is not present at all, fewer detectable labels (e.g., essentially none) are captured at the detection zone. The decreased abundance (e.g., absence) of captured detectable labels is indicative of a reduced level (e.g., absence) of the analyte in the sample.

In some embodiments, the devices are configured so that the presence of the detectable labels in the detection capture zone is determined visually (e.g., by the unaided human eye). The devices maybe configured so that the visually determined appearance of any amount of the color is indicative of the presence of the analyte (e.g., the presence of the analyte above a predetermined threshold). The absence of the color is indicative of the absence of the analyte (e.g., indicative that the analyte is not present in an amount that exceeds the predetermined threshold).

While conjugates detection capture zones have been described as capturing conjugates 507 by binding a linker of the conjugates, other embodiments are possible. For example, a conjugate may include a binding member by which the conjugate is captured in a detection capture zone but which does not bind the analyte present in the liquid sample.

A variation of the assay method involves a step of applying a liquid sample to a test device having one or more of the following features: a matrix for supporting the liquid sample flowing thereon comprising: (a) a blocking zone comprising a blocking-zone binding agent, wherein the blocking-zone binding agent comprises an analyte mimic immobilized on the blocking zone, wherein said analyte in said liquid sample and said analyte mimic compete for binding to a conjugate when present, wherein binding affinity between the analyte mimic and the conjugate is higher than that between the analyte and the conjugate; (b) a detection zone comprising, immobilized thereon, a detection-zone binding agent that exhibits binding specificity to said conjugate.

The sample is added to the mobilized zone comprising mobilizeable labeled conjugates comprising an analogue antibody. The application of the liquid sample effects competitive binding of the analyte and the conjugate for the blocking-zone binding agent.

A ligand which has a higher association constant than the analyte for the analogue antibody is typically immobilized at blocking zone. All of labeled antibody in the absence of analyte, which has moved to blocking zone, can bind to immobilized ligands. In the presence of analyte, the labeled analogue antibody will partially or entirely bind analyte, depending on the concentration of analyte in the sample. Some, most, or all of the analyte-bound labeled analogue antibodies do not bind at the blocking zone, and instead can move to a capture line or capture zone and be captured by an anti-antibody antibody or any other binding agent immobilized at capture zone

The method further comprises the step of determining the presence of said conjugate bound to said blocking-zone binding agent and/or said detection-zone binding agent, thereby detecting the presence of said analyte in said liquid sample.

### EXAMPLES

The following are non-limiting examples. Examples 1-7 describe the preparation and testing of a positive read morphine assay device. Examples 8-12 describe the preparation and testing of a positive read cocaine assay device. Examples 13-17 describe the preparation and testing of a positive read multi-analyte (THC/COC/MOR) assay device. Example 18 describes the method of comparison of the affinities of a MOR antibody for a MOR-BSA conjugate and for MOR

### Example 1

### Preparation of Colloidal Gold Particles

1.64 ml of 10% gold chloride solution (HAuCl₄ in dH₂O) was added to 1 liter of H₂O which was heated to 90° C. The solution was kept stirring and heated until the water reached boiling point. Then 1 ml of 23.6% sodium citrate (C₆H₅Na₃O₇ in dH₂O) was added to the solution and the solution was kept stirring for 5 minutes.

### Example 2

### Preparation of a Morphine Colloidal Gold Conjugate

A morphine-colloidal gold conjugate was prepared by adding 6.0 ml of phosphate buffer (0.1M, pH 6.5) drop wise with rapid stirring to 60 ml of the colloidal gold solution from Example 1. 0.6 ml of morphine-BSA conjugate (from Genclone) that had been diluted to 1 mg/ml with phosphate buffer (0.1 M, pH 6.5) was added quickly to the colloidal gold solution while stirring rapidly (about 750 rpm). The solution was kept stirring slowly (about 200 rpm) for 30 minutes at room temperature. Then 0.6 ml of 10% polyethylene glycol (PEG: MW 15,000 in dH₂O) was added quickly to the solution. The solution was kept stirring slowly for 30 minutes at room temperature. The colloidal gold solution was centrifuged at 30,000 g for 30 minutes at 4° C. The supernatant was discarded, and the pellet was suspended with 60 ml of 0.01 M, pH6.5 phosphate+0.05% casein buffer. The solution was centrifuged again. The supernatant was discarded and the pellet was suspended with 0.6 ml of PSC buffer (0.01 M phosphate, pH 7.0+2.5% sucrose+0.2% casein) to form a conjugate solution.

### Example 3

### Preparation of the Dry Conjugate Pad

A glass fiber pad (0.6 cm×30 cm) was treated with a solution containing 1% Rhodasurf+1% PVP+0.1% sodium casein+0.02% sodium azide in 0.5M sodium phosphate pH 7. The treated glass fiber pad was dried overnight. 0.01 ml of the conjugate solution from Example 2 was added to 1.19 ml of PSC buffer. The 1.2 ml of diluted conjugate solution was spread on a treated glass fiber pad. The wet glass fiber pad containing the conjugate was dried in a 44° C. oven for 2 hours. The dry gold conjugate pad was stored in a plastic bag with desiccant.

### Example 4

### Preparation of a Test Strip with a First Capture Zone

An antibody to morphine analogue was diluted at 1.8 mg/ml in 0.01M PBS. The diluted antibody solution was printed on a nitrocellulose membrane (Whatman GmbH) as double lines each 1.5-2.2 mm wide using a dispenser.

### Example 5

### Preparation of a Test Strip with Detection Capture Zone

A sheep antibody to BSA (1 mg/ml, from Immunology Consultants Laboratory, Inc) was printed as one line 0.8-1.2 mm wide on the membrane from Example 4. The sheep antibody line was spaced apart along the length of the membrane from the double lines formed in Example 4. The membrane was incubated at 37° C. for 24 hours.

### Example 6

### Preparation of a Sample Pad

A sample pad treatment solution was prepared by dissolving 4 g of PVP (Sigma, MW 10,000) and 0.8 g of Rhodasurf with 0.01M phosphate buffer, pH 7 to 100 ml dH₂O. 3.5 ml of the solution was applied to one strip of sample pad (1.8×30 cm glass fiber Grade 8964, Ahlstrom). The treated sample pad was dried at room temperature overnight.

### Example 7

### Positive Read Morphine Assay Devices

The sample pad from Example 6, the conjugate pad from Example 3, and the membrane strip from Example 5 were positioned so that an end of the sample pad overlaid an end of the conjugate pad and the opposite end of the conjugate pad overlaid the end of the membrane strip that was closer to the first capture zone (Example 4) than to the detection capture zone (Example 5). An absorbent pad was positioned in contact with the end of the membrane strip that was opposite the conjugate pad. A cardboard card was used to maintain the position of the assay device pieces.

The card with pieces was cut into strips, each having a portion of sample pad, conjugate pad, membrane strip, and absorbent pad. The strips were each positioned in a respective cassette formed of a water impermeable plastic. Each cassette included an opening which provided liquid access to the sample pad portion and an opening which provided visual access to the detection zone but not to the first capture zone.

The strips in cassettes were tested by applying 100 μl of sample to the sample pads of each strip. The samples were spiked with varying concentrations of morphine. The sample traveled through the conjugate pad where it mobilized the dry conjugate. A mixture of sample with mobilized conjugate traveled to the membrane strip, through the blocking zone and through the detection zone to the absorbent pad. For those samples having a morphine concentration higher than 300 ng/ml, a colored line appeared in the detection capture zone. For those samples having a morphine concentration less than 300 ng/ml, a colored line did not appear in the detection capture zone.

### Example 8

### Preparation of a Cocaine Colloidal Gold Conjugate

A COC-colloidal gold conjugate was prepared by adding 6.0 ml of phosphate buffer (0.1M, pH 5.8) drop wise with rapid stirring to 60 ml of colloidal gold from Example 1. 0.6 ml of benzoylecgonine-BTG conjugate (from Immunetics) diluted to 1 mg/ml with phosphate buffer (0.1 M, pH 5.8) was added quickly to the colloidal gold while stirring rapidly. The solution was kept stirring slowly for 30 minutes at room temperature. Then 0.6 ml of 2% casein was added quickly to the solution. The solution was kept stirring slowly for 30 minutes at room temperature. The colloidal gold solution was centrifuged at 30,000 g for 30 minutes at 4° C. The supernatant was discarded and the pellet was suspended with 60 ml of 0.01 M, pH5.8 phosphate, 0.05% casein buffer. The solution was centrifuged again. The supernatant was discarded and the pellet was suspended with 0.6 ml of PSC buffer (0.01 M phosphate, pH 7.0, 2.5% sucrose, 0.2% casein).

### Example 9

### Preparation of a Dry Conjugate Pad

A glass fiber pad (0.6 cm×30 cm) was treated with a solution containing 1% Rhodasurf+0.1% PVP+0.1% sodium casein+0.02% sodium azide in 0.5M sodium phosphate pH 7. The treated glass fiber pad was dried overnight. 0.012 ml of the conjugate solution from Example 8 was added to 1.188 ml of PSC buffer. The 1.2 ml of diluted conjugate solution was spread on a treated glass fiber pad. The wet glass fiber pad containing cocaine gold conjugate was put in 44° C. dryer for 2 hours. The dry COC gold conjugate pad was stored in a plastic bag with desiccant.

### Example 10

### Preparation of a Test Strip with First Capture Zone

A COC analogue antibody (from Omega) was diluted to 1.8 mg/ml with 0.01M PBS. The diluted antibody solution was printed on a nitrocellulose membrane (Whatman GmbH) as double lines each 1.5-2.2 mm wide using a dispenser.

### Example 11

### Preparation of a Test Strip with Detection Capture Zone

A mouse antibody to bovine-thromboglobulin (BTG) (1 mg/ml, from ABR-Affinity BioReagents Inc) was printed as one line 0.8-1.2 mm wide on the membrane from Example 10. The mouse antibody line was spaced apart along the length of the membrane from the double lines formed in Example 10. The coated membrane was incubated at 37° C. for 24 hours.

### Example 12

### Positive Read Cocaine Assay Devices

A sample pad prepared according to Example 6, the conjugate pad from Example 9, and the membrane strip from Example 10 were positioned so that an end of the sample pad overlaid an end of the conjugate pad and the opposite end of the conjugate pad overlaid the end of the membrane strip that was closer to the first capture zone (Example 9) than to the detection capture zone (Example 10). An absorbent pad was positioned in contact with the end of the membrane strip that was opposite the conjugate pad. A cardboard card was used to maintain the position of the assay device pieces.

The card with pieces was cut into strips, each having a portion of sample pad, conjugate pad, membrane strip and absorbent pad. The strips were each positioned in a respective cassette formed of a water impermeable plastic. Each cassette included an opening which provided liquid access to the sample pad portion and an opening which provided visual access to the detection zone but not to the blocking zone.

The strips in cassettes were tested by applying 100 μl of sample to the sample pads of each strip. The samples were spiked with varying concentrations of cocaine. The sample traveled through the conjugate pad where it mobilized the dry conjugate. A mixture of sample with mobilized conjugate traveled to the membrane strip, through the blocking zone and through the detection capture zone to the absorbent pad. For those samples having a cocaine concentration higher than 50 ng/ml, a colored line appeared in the detection capture zone. For those samples having a cocaine concentration less than 50 ng/ml, a colored line did not appear in the detection capture zone.

### Example 13

### Preparation of THC-Colloidal Gold Conjugate

A THC-colloidal gold conjugate was prepared by adding 6.0 ml of phosphate buffer (0.1M, pH 7.2) drop wise to 60 ml of colloidal gold with rapid stirring. 0.6 ml of THC— keyhole limpet hemocyaninconjugate (KLH) (Genclone) diluted to 1 mg/ml with phosphate buffer (0.1 M, pH 6.5) was added quickly to the colloidal gold while stirring rapidly. The solution was stirred slowly for 30 minutes at room temperature. Then 0.6 ml of 2% casein was added quickly to the solution. After the casein addition, the solution was stirred slowly for 30 minutes at room temperature. The colloidal gold solution was centrifuged at 30,000 g for 30 minutes at 4° C. The supernatant was discarded and the pellet was suspended with 60 ml of 0.01 M, pH7.2 phosphate, 0.05% casein buffer. The solution was centrifuged again. The supernatant was discarded and the pellet was suspended with 0.6 ml of PSC buffer.

### Example 14

### Preparation of a Dry Conjugate Pad

0.012 ml of the THC-gold conjugate from Example 13, 0.012 ml of COC-gold conjugate prepared in accord with Example 8, and 0.01 ml of MOR-gold conjugate prepared in accord with Example 2 were added to 1.166 ml of PSC buffer. The 1.2 ml of diluted conjugate mixture solution was spread on a fiberglass pad (0.6 cm×30 cm) that had been treated in accord with Example 3. The wet fiberglass pad containing THC, COC and MOR gold conjugates was put in 44° C. dryer for 2 hours. The dry THC, COC and MOR gold conjugate pad was stored in a plastic bag with desiccant.

### Example 15

### Preparation of a Test Strip with Multiple Capture Zones

THC analogue, COC analogue and MOR analogue antibodies were diluted at 1.8 mg/ml with 0.01M PBS. Each diluted antibody solution was printed as double line with a width of 1.5-2.2 mm on membrane (from Whatman GmbH) for each line using the ABON dispenser. Each line was spaced apart from the other lines. The lines were positioned so that, in use, liquid sample would sequentially encounter the capture zones corresponding to the THC analogue antibody, the COC analogue antibody, and then the MOR analogue antibody.

### Example 16

### Preparation of a Test Strip with Multiple Detection Capture Zones

KLH, BTG and BSA antibody were diluted at 1.0 mg/ml with 0.01M PBS. Each diluted antibody solution was printed as one line with a width of 0.8-1.2 mm on membrane for each line. The lines were positioned so that, in use, liquid sample would sequentially encounter the detection capture zones corresponding to the anti-KLH antibody (for THC detection), the anti-BTG antibody (for COC detection) and then the anti-BSA antibody (for MOR detection). The coated membrane was incubated at 37° C. for 24 hours.

### Example 17

### Positive Read Multi-Analyte Assay Devices

A sample pad prepared according to Example 6, the conjugate pad from Example 14, and the membrane strip from Example 16 were positioned so that an end of the sample pad overlaid an end of the conjugate pad and the opposite end of the conjugate pad overlaid the end of the membrane strip that was closer to each capture zone (Example 15) than to the corresponding detection capture zone (Example 16). An absorbent pad was positioned in contact with the end of the membrane strip that was opposite the conjugate pad. A cardboard card was used to maintain the position of the assay device pieces.

The card with pieces was cut into strips, each having a portion of sample pad, conjugate pad, membrane strip and absorbent pad. The strips were each positioned in a respective cassette formed of a water impermeable plastic. Each cassette included an opening which provided liquid access to the sample pad portion and an opening which provided visual access to the detection zones but not to the blocking zones.

The strips in cassettes were tested by applying 100 μl of sample to the sample pads of each strip. The samples were spiked with varying concentrations of cocaine, morphine, and THC. The sample traveled through the conjugate pad where it mobilized the dry conjugates. A mixture of sample with mobilized conjugates traveled to the membrane strip, through the blocking zones and through the detection zones to the absorbent pad. For those samples having a cocaine concentration higher than 300 ng/ml, a colored line appeared in the corresponding detection zone. For those samples having a cocaine concentration less than 300 ng/ml, a colored line did not appear in the detection zone. For those samples having a THC concentration higher than 50 ng/ml, a colored line appeared in the detection zone. For those samples having a THC concentration less than 50 ng/ml, a colored line did not appear in the corresponding detection zone. For those samples having a morphine concentration higher than 300 ng/ml, a colored line appeared in the corresponding detection zone. For those samples having a morphine concentration less than 300 ng/ml, a colored line did not appear in the corresponding detection zone.

### Example 18

### Comparison of the Affinities of a MOR Antibody for a MOR-BSA Conjugate and for MOR

1. ELISA well of strip, Lot# 002-91B, pre-coated MOR antibody (PN/LN: 1020003802/PO60308-2-0610 from Genclonn Inc)

2. MOR analyte: MOR solution (lot #002-13A) prepared at 1-ug/ml, diluted from MOR standard, PN/LN: 018033/0606000472, from Alltech Applied Science Inc.

3. MOR-BSA ligand: MOR-BSA (PN/LN: 1020001702/SMO 060426-2-0623, concentration at 5.22 mg/ml from Genclonn Inc.) diluted to 100 ug/ml

4. Secondary antibody: BSA antibody conjugated with HRP with concentration at 1 mg/ml from Immunology Consultants Laboratory Inc

5. TMB substrate: PN/LN 304176/060906 from Geogen Corp.

6. ELISA reader: Spectra Max (Plus 384) Molecular Device.

Method:

1. Control conditions—without competition with MOR analyte

Solution of MOR-BSA ligand, 100 μl per well, at concentration of 0 nM, 60 nM, 105 nM, 210 nM and 420 nM were added into each well pre-coated with anti-MOR antibody as a control group. The wells were incubated for 90 minutes at room temperature and washed three times with 0.01M PBS pH7.0. Solution of anti-BSA antibody conjugated with HRP, 100 μl per well, at concentration of 0.25 μg/ml was added into the wells. The wells were incubated for 90 minutes at room temperature and washed again as same as above. Solution of TMB substrate, 100 μl per well, was added into the wells. After the wells were incubated at room temperature for 10 minutes, 50 μl of 2N H₂SO₄ was added into each well to stop reaction. The color intensity of well was checked by ELISA reader at 450 nM wavelength.

2. Test condition—competition between MOR-BSA and MOR analyte

The MOR-BSA ligand and MOR analyte were mixed to make a final concentration at 402 nM for both of them in a solution. The solution was added into a well pre-coated with anti-MOR antibody as a competition well. The wells were incubated for 90 minutes at room temperature and washed three times by 0.01M PBS pH7.0. Solution of anti-BSA antibody conjugated with HRP, 100 μl per well, at concentration of 0.25 μg/ml was added into the wells. The wells were incubated for 90 minutes at room temperature and washed again as same as above.

Solution of TMB substrate, 100 μl per well, was added into the wells. After the wells were incubated at room temperature for 10 minutes, 50 μl of 2N H₂SO₄ was added into each well to stop reaction. The color intensity of well was checked by ELISA reader at 450 nM wavelength.

Results:

Control well

| MOR-BSA | | | | | |
|---|---|---|---|---|---|
| concentration | 0 nM | 60 nM | 105 nM | 210 nM | 420 nM |
| OD value | 0.171 | 0.399 | 0.449 | 0.580 | 0.729 |

Competition well

| MOR-BSA concentration | 420 nM |
|---|---|
| MOR concentration | 420 nM |
| OD value | 0.708 |

Discussion:

If the anti-MOR antibody binds with an equal affinity to the MOR-BSA ligand and to the MOR analyte, then only half of MOR-BSA ligand and half of MOR analyte added in a competition well will have the chance to bind to the antibody. In this scenario, we expect the OD of the competition well to be half of the OD of the control well. The resulting OD of the competition well will be closer to the OD value of the control well with 210 nM MOR-BSA.

If the anti-MOR antibody has a higher affinity to MOR-BSA than to the MOR analyte, then a higher concentration of the MOR-BSA will be bound to the MOR antibody. The resulting OD value will be closer to the OD value of the control well with 420 nM.

From the results obtained, the OD of the competition well (OD 0.708) was very close to OD of the control with 420 nM of MOR-BSA (OD 0.729). There appeared to be some competition between the MOR-BSA and MOR analyte in binding to the MOR antibody but the MOR-BSA appeared to have a higher affinity to the MOR antibody than the MOR analyte because of the slight decrease in OD compared to the control condition.

Conclusion:

According to this limited study, the MOR antibody from Genclonn appears to have a higher affinity to MOR-BSA than to the MOR analyte. The OD of competition well is higher than OD value of the control well with MOR-BSA at 210 nM but slightly lower than the OD of the control well with 420 nM.

1. A test device for detecting an analyte in a liquid sample, comprising; a matrix for supporting the liquid sample flowing thereon comprising:
(a) a blocking zone comprising a blocking-zone binding agent immobilized thereon, wherein said analyte in said liquid sample and a conjugate comprising an analyte mimic when present, compete for binding to said blocking-zone binding agent, wherein binding affinity between the analyte mimic and the blocking-zone binding agent is higher than that between the analyte and the blocking-zone binding agent;
(b) a detection zone comprising immobilized thereon a detection-zone binding agent that exhibits binding specificity to the conjugate comprising said analyte mimic.

2. The device of claim 1, wherein the conjugate further comprises a detectable label and the analyte mimic, wherein said label and mimic are linked via a linker.

3. The device of claim 1, wherein the blocking zone comprises a plurality of distinct blocking-zone binding agents, individual members of said plurality exhibit binding specificity to distinct analytes present in said sample.

4. The device of claim 1, wherein the detection zone comprises a plurality of distinct detection-zone binding agents, individual members of said plurality exhibit binding specificity to distinct conjugates present in said sample.

5. The device of claim 4, wherein each distinct conjugate comprises a detectable label and said analyte mimic, wherein said detectable label and said analyte mimic are linked via a linker, and further wherein the distinct conjugates are differentiated by one or more members selected from the group consisting of distinct linkers, distinct analyte mimics, and distinct detectable labels.

6. The device of claim 3, wherein said distinct blocking-zone binding agents are each immobilized to distinct regions.

7. The device of claim 4, wherein said distinct detection-zone binding agents are each immobilized to distinct regions.

8-10. (canceled)

11. The device of claim 1, wherein the matrix comprises a mobilization zone, said mobilization zone comprising the conjugate having a detectable label and the analyte mimic, wherein the conjugate is mobilizable upon application of said liquid sample.

12. The device of claim 11, wherein the mobilization zone comprises a plurality of distinct conjugates, wherein members of said plurality exhibit binding specificity to distinct blocking-zone binding agents and/or distinct detection-zone binding agents.

13. (canceled)

14. The device of claim 1, wherein the binding affinity between the analyte mimic and the blocking-zone binding agent is at least one fold higher than the binding affinity between the analyte and the blocking-zone binding agent, as measured by association constants.

15. A test device for detecting an analyte in a liquid sample, comprising;
a matrix for supporting the liquid sample flowing thereon comprising:
(a) a blocking zone comprising a blocking-zone binding agent, wherein the blocking-zone binding agent comprises an analyte mimic immobilized on the blocking zone, wherein said analyte in said liquid sample and said analyte mimic compete for binding to a conjugate when present, wherein binding affinity between the analyte mimic and the conjugate is higher than that between the analyte and the conjugate;
(b) a detection zone comprising, immobilized thereon, a detection-zone binding agent that exhibits binding specificity to said conjugate.

16. The device of claim 15, wherein the conjugate further comprises a detectable label and a moiety that exhibits binding specificity to said analyte and said analyte mimic, said label and said moiety are linked via a linker.

17. The device of claim 15, wherein the blocking zone comprises a plurality of distinct analyte mimics, individual members of said plurality of analyte mimics exhibit binding specificity to distinct conjugates present in said sample.

18. The device of claim 15, wherein the detection zone comprises a plurality of distinct detection-zone binding agents, individual members of said plurality exhibit binding specificity to distinct conjugates present in said sample.

19. The device of claim 18, wherein each distinct conjugate comprises a detectable label and the moiety, said detectable label and said moiety are linked via a linker, and further wherein the distinct conjugates are differentiated by one or more members selected from the group consisting of distinct linkers, distinct moieties, and distinct detectable labels.

20. The device of claim 17, wherein said distinct analyte mimics are each immobilized to distinct regions.

21. The device of claim 18, wherein said distinct detection-zone binding agents are each immobilized to distinct regions.

22-24. (canceled)

25. The device of claim 15, wherein the matrix comprises a mobilization zone, said mobilization zone comprising the conjugate having a detectable label and the moiety, wherein the conjugate is mobilizable upon application of said liquid sample.

26. (canceled)

27. The device of claim 25, wherein the mobilization zone comprises a plurality of distinct conjugates, wherein members of said plurality of distinct conjugates exhibit binding specificity to distinct analytes, distinct analyte mimics, and/or distinct detection-zone binding.

28. The device of claim 15, wherein the binding affinity between the analyte mimic and the conjugate is at least one fold higher than that between the analyte and the conjugate, as measured by association constants.

29-44. (canceled)
